(19) United States
(12) Patent Application Publication
(10) Pub. No.: US 2016/0214911 A1
(43) Pub. Date: Jul. 28, 2016

(54) DUAL RISER FLUID BED PROCESS AND REACTOR

(71) Applicant: Kellogg Brown & Root LLC, Houston, TX (US)

(72) Inventor: Curtis ENG, Sugar Land, TX (US)

(21) Appl. No.: 15/056,020

(22) Filed: Feb. 29, 2016

Related U.S. Application Data
(63) Continuation of application No. 13/802,302, filed on Mar. 13, 2013, now abandoned.

(51) Int. Cl. C07C 1/20; C07C 4/06

(52) U.S. Cl. CPC C07C 1/20; C07C 4/06; C07C 2529/40

(57) ABSTRACT

Processes and systems for cracking feeds to produce olefins are provided. The process for cracking feeds can include converting a first feed containing at least about 50 wt % methanol in a first riser under a first set of process conditions to produce a first effluent enriched in ethylene, propylene, or a mixture thereof, wherein the first effluent contains at least about 25 wt % dry basis propylene and converting a second feed containing C₄-C₁₀ light hydrocarbons in a second riser under a second set of process conditions to produce a second effluent enriched in ethylene, propylene, or a mixture thereof. The process can also include combining the first effluent with the second effluent to produce a mixed effluent, separating the mixed effluent to produce a coked-catalyst and a gaseous product, regenerating the coked-catalyst, and recycling the regenerated catalyst to the first and second risers.

### CROSS-REFERENCE TO RELATED APPLICATIONS

This application is a continuation of co-pending U.S. patent application having Ser. No. 13/802,302, filed on Mar. 13, 2013, which is incorporated by reference herein.

### BACKGROUND

1. Field

Embodiments described herein generally relate to processes utilizing fluidized bed reactors. More particularly, such embodiments relate to processes and dual riser reactors for converting light hydrocarbons to olefins in the presence of a fluidized bed.

2. Description of the Related Art

Fluid Catalytic Cracking (FCC) is a process used in refineries to improve yields for transportation fuels such as gasoline and distillates. The FCC process uses a reactor called a riser, essentially a pipe, in which a hydrocarbon feed is contacted with catalyst particles to effect the conversion of the feed to more valuable products. The FCC unit converts gas oil feeds by “cracking” the hydrocarbons into smaller molecules. The resulting hydrocarbon gas and catalyst mixture both flow in the riser, hence the term fluid catalytic cracking.

The cracking reaction is endothermic, meaning that heat must be supplied to the reactor to heat the feedstock and maintain sufficient reaction temperature. During the conversion of heavy feeds (such as vacuum gas oils, reduced crudes, atmospheric tower bottoms, vacuum tower bottoms, and the like), coke is formed. The coke is deposited on the catalyst and ultimately burned with an oxygen source such as air in a regenerator. Burning the coke is an exothermic process that can supply the heat needed for the cracking reaction. In balanced operation, no external heat source or fuel is needed to supplement the heat from coke combustion. Should a heat imbalance exist, such as making too much coke and generating excessive heat for the reactions, it is possible to use a catalyst cooler and/or other process modifications in mitigation, especially with heavy feeds or high severity operation. Unlike heavy feeds, light feeds (such as light cracked naphtha) do not make enough coke to maintain heat balance in the FCC unit. Thus, an external source of heat input is required to keep the FCC unit in heat balance when using predominantly light feeds. Adding external heating and/or cooling sources to an FCC unit can increase the capital and operational expenditures of the process.

There is a need, therefore, for more efficient processes and systems for cracking heavy feeds and light feeds with a reduced need for external cooling and/or heating sources.

### BRIEF DESCRIPTION OF THE DRAWINGS

FIG. 1 depicts an illustrative dual riser fluidized catalytic cracking (FCC) reactor for processing one or more hydrocarbons, according to one or more embodiments described.

FIG. 2 depicts an illustrative block process flow diagram incorporating the dual riser FCC reactor depicted in FIG. 1 and further having one or more recycle lines from downstream process units, according to one or more embodiments described.

### DETAILED DESCRIPTION

Processes and systems for cracking feeds to produce olefins are provided. The process for cracking feeds can include a dual riser fluidized catalytic cracking process including converting a first feed containing at least about 50 wt % methanol in a first riser under a first set of process conditions to produce a first effluent enriched in ethylene, propylene, or a mixture thereof, wherein the first effluent contains at least about 25 wt % dry basis propylene. The process can further include converting a second feed containing C₄-C₁₀ light hydrocarbons in a second riser under a second set of process conditions to produce a second effluent enriched in ethylene, propylene, or a mixture thereof. The process can also include combining the first effluent with the second effluent to produce a mixed effluent, separating the mixed effluent to produce a coked-catalyst and a gaseous product, regenerating the coked-catalyst by combusting the coke in a regenerator to produce a regenerated catalyst, and recycling the regenerated catalyst to the first and second risers.

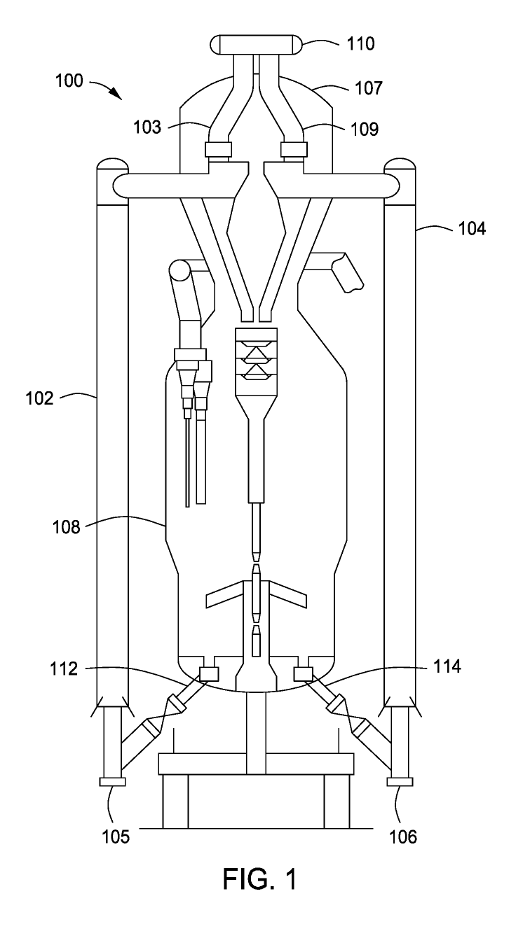

FIG. 1 depicts an illustrative dual riser fluid catalytic cracking (FCC) system 100 for processing one or more hydrocarbons, according to one or more embodiments. The FCC system 100, also referred to as a FCC reactor, can include one or more first risers 102, one or more second risers 104, one or more catalyst separation or disengagement zones 107, and one or more catalyst regeneration zones 108. A first feed via inlet 105 can be introduced to the first riser 102 under first-riser conditions to form a first effluent via line 103 enriched in ethylene, propylene or a mixture thereof. The first feed via inlet 105 can be or include methanol. A second feed via inlet 106 can be introduced to the second riser 104 under second-riser conditions to form a second effluent via line 109 enriched in ethylene, propylene or a mixture thereof. The second feed via inlet 106 can be or include one or more light hydrocarbons. The first and second feeds can be different from one another. The first-riser and second-riser conditions can be independently selected to favor production of ethylene, propylene or a mixture thereof.

The ethylene and/or propylene yields can be increased in a process that employs a single converter and dual risers, i.e., a dual riser fluid bed reactor, for converting methanol in the first riser and C₄+ olefins in the second riser. By use of the dual riser fluid bed reactor, methanol can be converted to predominately ethylene and/or propylene in the first riser (methanol to olefins or “MTO”) and C₄+ hydrocarbon byproducts can be recycled and converted to ethylene and/or propylene in the second riser. Further, the heat generated by the MTO process in the first riser can supply at least a portion of or all the heat necessary for the conversion of the C₄+ hydrocarbon byproducts in the second riser, thus maintaining or improving a balance of heat in the overall process. In this manner, total ethylene and/or propylene yields can be significantly increased compared to current fixed bed MTO processes. By segregating feeds to the risers, each feed can be processed at conditions that optimize olefin production. For different feeds, the appropriate riser conditions may be different. For example, with segregated methanol and olefinic light hydrocarbon feeds, the riser receiving the methanol feed can have a different temperature, catalyst-to-feed ratio, partial pressure, residence time, flow rate, catalyst, and/or other process condition(s) as compared to the riser to which the olefinic feed is supplied.

As noted above, the first feed in line 105 can be or include methanol. As such, the first feed in line 105 can also be referred to as a “methanol feed” or a “methanol containing feed.” The first feed in line 105 can be obtained from any source or combination of sources. In some embodiments, the first feed can be a byproduct from the production of syngas. In one or more embodiments, the first feed in line 105 can optionally include one or more of ethers, oxygenates, and/or other oxygenated feeds. The first feed can contain methanol in any amount. For example, the first feed can have a methanol content in an amount from a low of about 10 wt %, about 15 wt %, about 25 wt %, about 35 wt %, or about 45 wt % to a high of about 50 wt %, about 65 wt %, about 75 wt %, about 85 wt %, about 95 wt %, or about 100 wt %. The first feed can also be referred to as a “crude” methanol stream or a purified methanol stream when having a methanol concentration from about 70 wt % to about 99 wt % or more, about 75 wt % to about 98 wt %, or about 85 wt % to about 95 wt %. The first feed can also contain one or more ethers, one or more oxygenates, or any mixture thereof in any amount(s). For example, the first feed can contain ethers and/or oxygenates in an amount from a low of about 1 wt %, about 10 wt %, or about 20 wt % to a high of about 35 wt %, about 50 wt %, or about 60 wt %. Illustrative oxygenates can include, but are not limited to, ethanol, iso-propanol, n-propanol, iso-butanol, n-butanol, ketones, aldehydes, organic acids, ethers, or any mixture or combination thereof. Illustrative ethers can include, but are not limited to, dimethyl ether, methyl tertiary butyl ether (MTBE), ethyl tertiary butyl ether, tertiary amyl methyl ether (TAME), tertiary amyl ethyl ether, or any mixture or combination thereof.

As noted above, the second feed can be or include any hydrocarbon or combination of hydrocarbons having four or more carbon atoms (C₄+ hydrocarbons). As such, the second feed can also be referred to as a “C₄+ feed” or a “C₄+ hydrocarbon containing feed.” The second feed can be or include any paraffinic or olefinic hydrocarbon having four or more carbon atoms. Illustrative hydrocarbon compounds that can be present in the second feed can include, but are not limited to, paraffinic, cycloparaffinic, monoolefinic, diolefinic, cycloolefinic, naphthenic, aromatic hydrocarbons, hydrocarbon oxygenates, or any mixture or combination thereof Additional hydrocarbons that can be present in the second feed can include, but are not limited to, light paraffinic naphtha (naphtha limited to hydrocarbon molecules having less than 12 carbon atoms and at least 80 wt % paraffins, no more than 10 wt % aromatics, and no more than 40 wt % cycloparaffins), heavy paraffinic naphtha (naphtha having hydrocarbon molecules with 12 carbon atoms and at least 80 wt % paraffins, no more than 10 wt % aromatics, and no more than 40 wt % cycloparaffins), light olefinic naphtha (naphtha limited to hydrocarbon molecules having less than 12 carbon atoms and at least 20 wt % olefins), heavy olefinic naphtha (naphtha having hydrocarbon molecules with 12 carbon atoms and at least 20 wt % olefins), mixed paraffinic C₄ hydrocarbons, mixed olefinic C₄ hydrocarbons (such as raffinates), mixed paraffinic C₅ hydrocarbons, mixed olefinic C₅ hydrocarbons (such as raffinates), mixed paraffinic and cycloparaffinic C₆ hydrocarbons; non-aromatic fractions from an aromatics extraction unit; oxygenate-containing products from a Fischer-Tropsch unit; or the like; or any mixture or combination thereof. In addition to the C₄+ hydrocarbons, the second feed can also include one or more oxygenates having from 1 to 4 carbon atoms, ethers having from 2 to 8 carbon atoms, or any mixture or combination thereof.

The second feed in line 106 can have a concentration of the C₄+ hydrocarbons from a low of about 15 wt %, about 20 wt %, about 25 wt %, about 35 wt %, or about 45 wt % to a high of about 85 wt %, about 90 wt %, about 95 wt %, about 99 wt %, or about 99.99 wt %. For example, the second feed in line 106 can have a C₄+ hydrocarbon concentration from about 25 wt % to 100 wt %, about 45 wt % to about 99 wt %, or about 55 wt % to about 95 wt %. In an example, the second feed in line 106 can contain less than 25 wt %, less than 20 wt %, less than 15 wt %, less than 10 wt %, less than 5 wt %, less than 3 wt %, or less than 1 wt % hydrocarbon compounds having less than 4 carbon atoms.

One or more catalysts suitable for the conversion of methanol to olefins can be introduced via line 112 in the first riser 102 and via line 114 to the second riser 104. The catalyst introduced via lines 112, 114 to the first riser 102 and the second riser 104, respectively, can be one that favors the production of propylene and/or ethylene from the feeds introduced thereto. For the cracking of light feeds, zeolite catalysts can be used alone or in conjunction with other known catalysts useful in fluidized catalytic cracking (such as crystalline zeolite molecular sieves, containing both silica and alumina with other modifiers such as phosphorous). Illustrative catalysts can include, but are not limited to, ZSM-5 and/or similar catalysts, Y-type zeolites, USY, REY, RE-USY, silicoaluminophosphate (SAPO) molecular sieves, faujasite and other synthetic and naturally occurring zeolites and mixtures thereof. For example, synthetic aluminosilicate zeolite catalysts such as ZSM-5 can be used in one embodiment, while in other embodiments SAPO molecular sieves such as SAPO-34 or SAPO-17 can be employed. Both of these types of zeolite catalysts can convert methanol to ethylene and propylene at a selectivity of about 70% to about 80% or more, with SAPO-34 forming about equal amounts of ethylene and propylene while ZSM-5 can favor the formation of propylene over ethylene in approximately a 4:1 ratio. In one or more embodiments, ZSM-5 can be used to convert methanol to olefins in the first riser. Certain non-zeolite catalysts can also be employed, for example bi-functional supported acid-base catalysts such as tungsten oxide over alumina (WO₃/Al₂O₃). Further, an MTO catalyst can be blended with one or more other catalysts, for example on an alumina support. In one or more embodiments, the MTO catalyst can be included in the blend in minor amounts, such as from about 10 wt % to about 40 wt %, or from about 15 wt % to about 35 wt %, or from about 20 wt % to about 30 wt %, based on the total weight of catalyst.

The first riser 102 and the second riser 104 can include the same or different catalysts. In certain optional embodiments, both the first riser 102 and the second riser 104 can employ a zeolite catalyst, either alone or in combination with one or more other catalysts. When other catalysts are used, such other catalysts can be present in only the first riser 102, only the second riser 104, or in both the first riser 102 and the second riser 104, and when used in both the first riser 102 and the second riser 104 the additional catalysts can be the same or different. The catalyst particles can include catalysts selected from ZSM-5, SAPO-34, and SAPO-17 and any mixture thereof. In one or more embodiments, both of the first and second risers can employ a catalyst containing ZSM-5.

The catalytic cracking processes can include contacting the catalyst directly with the feed, forming a catalytically cracked product containing cracked hydrocarbons and coked catalyst. The coked catalyst can be separated from the catalytically cracked product within the disengagement zone 107. At least a portion of the hydrocarbon that remains with the separated coked catalyst can be removed. The coked catalyst can be introduced to the catalyst regeneration zone or catalyst regenerator 108 where at least a portion of the carbon or coke contained on/in the catalyst can be combusted to produce heat and regenerated catalyst. The regenerated catalyst can be recycled to the first riser 102 and/or the second riser 104.

The first feed in line 105 and the second feed in line 106 can be introduced to the first riser 102 and the second riser 104, respectively, at a first feed to second feed weight ratio from a low of about 1:10, about 1:5, about 1:4, about 1:3, about 1:2 to a high of about 2:1, about 3:1, about 4:1, about 5:1, about 10:1. For example, the first feed to second feed weight ratio can be from about 1:5 to about 5:1, from about 1:2 to about 2:1, from about 2:3 to about 3:2, from about 4:5 to about 5:4, or about 9:10 to about 10:9. In one or more embodiments, the first feed in line 105 can include about 10 wt % or more methanol and the second feed in line 106 can include about 20 wt % or more C₄+ hydrocarbons and the first and second feeds can be introduced to the first and second risers 102, 104, respectively, at a first feed to second feed weight ratio of about 1:1 to about 10:1.

The catalyst to first feed weight ratio within the first riser 102 can be from a low of about 4:1, about 8:1, or about 15:1 to a high of about 25:1, about 40:1, or about 50:1. Similarly, the second riser 104 can include any catalyst-to-first feed ratio. For example, the second riser 104 can include a catalyst to feed ratio from a low of about 5:1, about 8:1, or about 10:1 to a high of about 32:1, about 35:1, or about 45:1. In one or more embodiments, the first feed in line 105 can include about 10 wt % or more methanol, the catalyst can be or include ZSM-5 catalyst, and the catalyst to first feed ratio can range from about 20:1 to about 30:1. The second feed in line 106 can also include the include ZSM-5 catalyst, and the catalyst to first feed ratio can range from about 12:1 to about 25:1.

The first riser 102 can be operated at a temperature from a low of about 200° C., about 300° C., or about 350° C. to a high of about 375° C., about 400° C., or about 450° C. For example, the first riser 102 can be operated at a temperature of about 250° C. to about 425° C., about 315° C. to about 390° C., or about 325° C. to about 360° C. The first riser 102 can be operated at a pressure from a low of about 140 kPa, about 200 kPa, or about 250 kPa to a high of about 300 kPa, about 350 kPa, or about 400 kPa. For example, the first riser 102 can be operated at a pressure of about 150 kPa to about 310 kPa, about 165 kPa to about 225 kPa, or about 175 kPa to about 200 kPa. The first feed in the first riser 102 can have a residence time from a low of about 0.1 second (s), about 0.5 s, or about 1 s to a high of about 2 s, about 5 s, or about 10 s. For example, the first feed in the first riser 102 can have a residence time of about 0.2 s to about 8 s, about 0.7 s to about 4 s, or about 1.5 s to about 2.5 s. The first feed introduced via inlet 105 to the first riser 102 can be preheated from waste heat provided from downstream process fractionation steps including, but not limited to, main fractionator pumparound systems. The first feed in line 105 can be preheated to a temperature ranging from about 35° C. to about 100° C., but can be preheated up to about 350° C. and supplied to the riser as vapor or a two-phase mixed vapor and liquid stream.

The second riser 104 can be operated at a temperature from a low of about 400° C., about 450° C., or about 500° C. to a high of about 675° C., about 750° C., or about 900° C. For example, the second riser 104 can be operated at a temperature of about 500° C. to about 725° C., about 475° C. to about 700° C., or about 525° C. to about 650° C. The second riser 104 can be operated under a pressure from a low of about 140 kPa, about 175 kPa, or about 225 kPa to a high of about 300 kPa, about 350 kPa, or about 400 kPa. For example, the second riser 104 can be operated at a pressure of about 150 kPa to about 350 kPa, about 200 kPa to about 325 kPa, or about 250 kPa to about 310 kPa. The second feed in the second riser 104 can have a residence time from a low of about 0.1 s, about 0.5 s, or about 1 s to a high of about 2 s, about 5 s, or about 10 s. For example, the second feed in the second riser 104 can have a residence time of about 0.2 s to about 8 s, about 0.7 s to about 4 s, or about 1.5 s to about 2.5 s. The second riser 104 can have a catalyst to second feed weight ratio from a low of about 5:1, about 10:1, or about 15:1 to a high of about 25:1, about 35:1, or about 40:1. The second feed introduced via inlet 106 to the second riser 104 can be preheated from waste heat provided from downstream process fractionation steps including, but not limited to, main fractionator pumparound systems. The second feed in line 106 can be preheated to a temperature ranging from about 90° C. to about 370° C., but can be preheated up to about 510° C. and supplied to the riser as vapor.

The second riser 104 can also include fluidized catalytic cracking of hydrocarbons in the C₄ to C₈ range to produce propylene. Feeds having relatively high olefin content, e.g., about 25 wt % or more olefins, can be introduced to the second riser. Thus, by-product C₄ and C₅ cuts from an olefins plant, either partially hydrogenated or as raffinate from an extraction process, can be feeds to the second riser 104. One benefit of the process can be the ability to process other potentially low value olefins-rich streams, such as FCC and coker light naphthas from the refinery. These feeds, in consideration of new motor gasoline regulations regarding vapor pressure, olefins content and oxygenate specifications, can have increasingly low value as blend stock for gasoline, but can be suitable feeds for the second riser 104. In addition to propylene, the process can also produce byproduct ethylene and a high octane, aromatic gasoline fraction that can add more value to the overall operating margin.

Further, FCC naphtha can be re-cracked in the presence of one or more zeolitic catalysts such as ZSM-5, with relatively high catalyst-to-feed ratios and high riser outlet temperatures, to produce olefins. To increase olefin yields from light olefinic feeds such as recycled cracked naphtha, the second riser 104 can operate at a riser outlet temperature of approximately 590° C. to 675° C.; from mixed olefinic C₄'s at a riser outlet temperature of approximately 550° C. to 650° C.; or from olefinic Cₛ's with a riser outlet temperature of approximately 650° C. to 675° C. The operating pressure for light olefinic feeds can range from about 40 kPa to about 700 kPa. For example catalyst-to-feed ratios for light olefinic feeds, measured in weight of catalyst to weight of hydrocarbon feed, can range from about 5:1 to about 70:1. In another example, catalyst-to-feed ratios for light olefinic feeds, measured in weight of catalyst to weight of hydrocarbon feed, can range from about 8:1 to about 50:1, from about 10:1 to about 25:1, or from about 12:1 to about 18:1.

The olefin yield from paraffinic feeds, e.g., non-aromatic C6-C8 hydrocarbons “raffinate” from an aromatic extraction unit, can be increased by operating the second riser 104 at a second riser 104 outlet temperature of approximately 620° C. to 720° C.; and from paraffinic feeds such as pentanes, at a second riser 104 outlet temperature of approximately 620° C. to 700° C. The operating pressure for paraffinic feeds can range from about 40 kPa to about 700 kPa. For example, catalyst-to-feed ratios for light paraffinic feeds can range from about 5:1 to about 80:1. In another example, catalyst-to-feed ratios for light paraffinic feeds, measured in weight of catalyst to weight of hydrocarbon feed, can range from about 12:1 to about 25:1.

The temperature and/or catalyst, e.g., ZSM-5, concentration level can, at least in part, cause the olefins and/or paraffins to crack. The riser outlet temperature and the heat of reaction can maximize the effectiveness of the catalyst.

Associated systems for the dual riser reactors 102, 104 can be standard FCC systems and can include air supply, flue gas handling and heat recovery. Reactor overheads can be cooled and washed to recover entrained catalyst, which can be recycled back to the reactor. The net overhead product can be routed to the primary fractionator in the olefins plant, although, depending on the available capacity in a given plant, the reactor effluent could alternately be further cooled and routed to an olefins plant cracked gas compressor, or processed for product recovery.

The second riser 104 in the dual riser unit can process a light feed in line 106 with a coke precursor, wherein the light feedstock is as described above and produces insufficient coke for heat balanced operation, and the coke precursor is present to supply sufficient coke to facilitate heat-balancing both risers, or at least to reduce the amount of supplemental fuel required for heat balancing. An advantage of using a heavy feedstock as a supplemental coke precursor is that some heavy oil can be produced to aid in fines recovery, replacing some or all of any supplemental import oil (such as fuel oil) that can be used in recovering fines from the light feed riser effluents. In one or more embodiments, the use of coke precursors and/or supplemental import oil is reduced or eliminated because the heat required for heat-balanced operation is provided by the exothermic methanol-to-olefins reaction in the first riser 102.

The process can further include recovering catalyst and separating gas from the first and second effluents, optionally in a common separation device such as the separation zone 107. The recovered catalyst can be regenerated from the first riser 102 and the second riser 104 by combustion of coke in a regenerator, or regeneration zone 108, to obtain hot, regenerated catalyst. The hot regenerated catalyst can be re-circulated to the first and second risers 102, 104 to sustain a continuous operating mode. In one or more embodiments, C₄+ products can be recycled to the second riser 104 to extinction, thus eliminating the need for a purge or “drag” stream to remove paraffins from the system.

The first effluent in line 103 and the second effluent in line 109 can be combined or otherwise mixed. For example, the first and second effluents can be recovered as a combined effluent via line 110. The combined effluent in line 110 can contain the first effluent in an amount from a low about 1 wt %, about 10 wt %, about 20 wt %, about 30 wt %, or about 40 wt % to about 60 wt %, about 70 wt %, about 80 wt %, about 90 wt %, or about 99 wt %. For example, the combined effluent in line 110 can contain the first effluent in amounts ranging from about 20 wt % to 80 wt %, about 30 wt % to about 70 wt %, about 40 wt % to about 60 wt %, or about 45 wt % to about 55 wt %. The combined effluent in line 110 can contain the second effluent in amounts ranging from a low about 1 wt %, about 10 wt %, about 20 wt %, about 30 wt %, or about 40 wt % to about 60 wt %, about 70 wt %, about 80 wt %, about 90 wt %, or about 99 wt %. For example, the combined effluent in line 110 can contain the second effluent in amounts ranging from about 20 wt % to 80 wt %, about 30 wt % to about 70 wt %, about 40 wt % to about 60 wt %, or about 45 wt % to about 55 wt %.

The dual riser process can, if desired, be integrated with one or more steam pyrolysis units. Integration of the catalytic and pyrolytic cracking units allows for flexibility in processing a variety of feedstocks. The integration allows thermal and catalytic cracking units to be used in a complementary fashion in a new or retrofitted petrochemical complex. The petrochemical complex can be designed to use the lowest value feed streams available. Integration can allow for production of an overall product slate with maximum value through routing of various by-products to the appropriate cracking technology.

The first effluent in line 103 can have an olefins concentration on a dry basis of at least about 65 wt %, at least about 75 wt %, at least about 85 wt %, or at least about 95 wt %. For example, the first effluent in line 103 can have an olefins concentration on a dry basis from a low of about 70 wt %, about 75 wt %, or about 80 wt % to a high of about 90 wt %, about 95 wt %, or about 99 wt %. The first effluent in line 103 can have an ethylene concentration on a dry basis of at least about 5 wt %, at least about 10 wt %, at least about 15 wt %, at least about 20 wt %, at least about 25 wt %, at least about 30 wt %, or at least about 40 wt %. For example, the first effluent in line 103 can have an ethylene concentration on a dry basis from a low of about 5 wt %, about 10 wt %, about 15 wt %, about 25 wt %, or about 35 wt % to a high of about 40 wt %, about 50 wt %, about 60 wt %, about 65 wt %, or about 70 wt %. The first effluent in line 103 can have a propylene concentration on a dry basis of at least about 15 wt %, at least about 20 wt %, at least about 25 wt %, at least about 35 wt %, at least about 45 wt %, at least about 55 wt %, or at least about 65 wt %. For example, the first effluent in line 103 can have a propylene concentration on a dry basis from a low of about 30 wt %, about 40 wt %, or about 45 wt % to a high of about 55 wt %, about 70 wt %, or about 80 wt %.

The first feedstock in line 105 can include at least about 50 wt % methanol, at least about 60 wt % methanol, at least about 70 wt % methanol, at least about 80 wt % methanol, at least about 90 wt % methanol, or at least about 95 wt % methanol and the first effluent in line 103 can include at least about 25 wt % dry basis propylene, at least about 35 wt % dry basis propylene, or at least about 40 wt % dry basis propylene. In one or more embodiments, the first feedstock in line 105 can include at least about 50 wt % methanol, at least about 60 wt % methanol, at least about 70 wt % methanol, at least about 80 wt % methanol, at least about 90 wt % methanol, or at least about 95 wt % methanol and the first effluent in line 103 can include at least about 25 wt % dry basis ethylene, at least about 35 wt % dry basis ethylene, or at least about 40 wt % dry basis ethylene. At least about 40 wt %, at least about 50 wt %, or at least about 60 wt % of the first feedstock in line 105 can be converted to ethylene, propylene, or a mixture thereof. For example, a low of about 45 wt %, about 55 wt %, or about 65 wt % to a high of about 75 wt %, about 85 wt %, or about 95 wt % of the first feedstock in line 105 can be converted to ethylene, propylene, or a mixture thereof. The first effluent in line 103 can have a combined propylene and ethylene concentration on a dry basis of at least about 50 wt %, at least about 60 wt %, or at least about 75 wt %, based on the total weight of the first effluent in line 103. For example, the first effluent in line 103 can have a combined propylene and ethylene concentration on a dry basis from a low of about 55 wt %, about 65 wt %, or about 70 wt % to a high of about 80 wt %, about 90 wt %, or about 99 wt %.

The second effluent in line 109 can have an olefins concentration on a dry basis of at least about 15 wt %, at least about 20 wt %, at least about 25 wt %, at least about 30 wt %, at least about 35 wt %, at least about 40 wt %, or at least about 45 wt %. For example, the second effluent in line 109 can have an olefins concentration on a dry basis from a low of about 18 wt %, about 27 wt %, about 33 wt %, about 42 wt %, or about 50 wt % to a high of about 65 wt %, about 75 wt %, about 85 wt %, or about 95 wt %. The second effluent in line 109 can have an ethylene concentration on a dry basis of at least about 5 wt %, at least about 10 wt %, at least about 15 wt %, at least about 20 wt %, or at least about 25 wt %. For example, the second effluent in line 109 can have an ethylene concentration on a dry basis from a low of about 8 wt %, about 12 wt %, about 18 wt %, or about 24 wt % to a high of about 50 wt %, about 65 wt %, about 75 wt %, or about 85 wt %. The second effluent in line 109 can have a propylene concentration on a dry basis of at least about 15 wt %, at least about 20 wt %, at least about 25 wt %, or at least about 30 wt %. For example, the second effluent in line 109 can have a propylene concentration on a dry basis from a low of about 12 wt %, about 18 wt %, about 28 wt %, or about 32 wt % to a high of about 60 wt %, about 75 wt %, about 85 wt %, or about 95 wt %. The second feedstock in line 106 can include at least about 50 wt % C₄-C₈ hydrocarbons, at least about 60 wt % C₄-C₈ hydrocarbons, at least about 70 wt % C₄-C₈ hydrocarbons, at least about 80 wt % C₄-C₈ hydrocarbons, or at least about 90 wt % C₄-C₈ hydrocarbons and the second effluent in line 109 can include at least about 10 wt % propylene, at least about 20 wt % propylene, at least about 25 wt % propylene, at least about 30 wt % propylene or at least about 40 wt % propylene and at least about 5 wt % ethylene, at least about 10 wt % ethylene, at least about 15 wt % ethylene, at least about 20 wt % ethylene, or at least about 25 wt % ethylene.

At least about 20 wt %, at least about 30wt %, at least about 40 wt %, or at least about 50 wt % of the second feed in line 106 can be converted to ethylene, propylene, or a mixture thereof. For example, a low of about 25 wt %, about 35 wt %, about 45 wt %, or about 55 wt % to a high of about 65 wt %, about 75 wt %, about 85 wt %, or about 90 wt % of the second feed in line 106 can be converted to ethylene, propylene, or a mixture thereof. At least about 15 wt %, at least about 25 wt %, at least about 35 wt %, at least about 45 wt %, or at least about 55 wt % of the second feed in line 106 can be converted to ethylene and propylene. For example, a low of about 20 wt %, about 30 wt %, about 40 wt %, or about 50 wt % to a high of about 60 wt %, about 75 wt %, about 85 wt %, or about 90 wt % of the second feed in line 106 can be converted to ethylene and propylene. At least about 10 wt %, at least about 12 wt %, at least about 14 wt %, at least about 16 wt %, at least about 18 wt %, at least about 20 wt %, at least about 22 wt %, at least about 24 wt %, or at least about 25 wt % of the second feed in line 106 can be converted to ethylene. For example, a low of about 15 wt %, about 20 wt %, or about 25 wt % to a high of about 35 wt %, about 45 wt %, or about 65 wt % of the second feed in line 106 can be converted to ethylene. At least about 15 wt %, at least about 17 wt %, at least about 19 wt %, at least about 21 wt %, at least about 23 wt %, at least about 25 wt %, at least about 27 wt %, at least about 29 wt %, or at least about 30 wt % of the second feed in line 106 can be converted to propylene. For example, a low of about 25 wt %, about 30 wt %, or about 35 wt % to a high of about 45 wt %, about 65 wt %, or about 75 wt % of the second feed in line 106 can be converted to propylene.

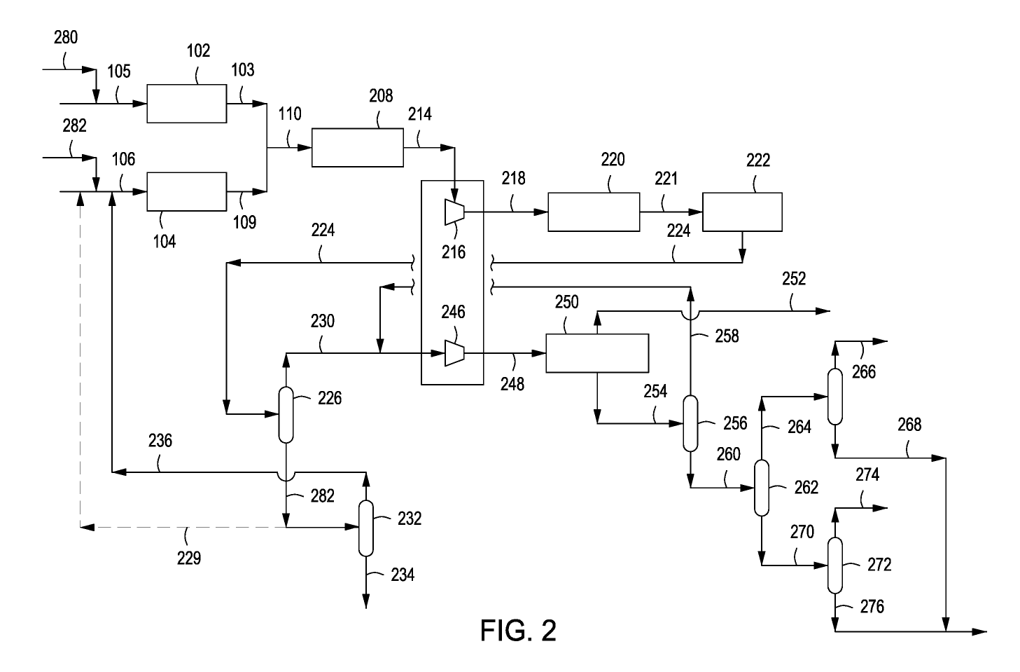

FIG. 2 depicts an illustrative block process flow diagram incorporating the dual riser FCC reactor depicted in FIG. 1 and further having one or more recycle lines from downstream process units, according to one or more embodiments. The embodiment depicted is one incorporating a dual-riser reactor having a first riser 102 and a second riser 104 fluidly coupled to a common catalyst regeneration zone 108, as exemplified in FIG. 1. The first riser 102 and the second riser 104 can receive respective first and second feed streams 105 and 106. In an embodiment, the first feed 105 includes methanol, and the second feed 106 includes C₄ to C₁₀ light hydrocarbons. In an embodiment, the second riser 104 can additionally be supplied with a recycle 229 of a bottom stream 228 recovered from a depropanizer 226 and/or an effluent or overhead stream 236 recovered from a gasoline splitter 232, as described below.

If desired, oxygenates can be fed to the first riser 102 via line 280, and suitable coke precursor can be fed to second riser 104 via line 282. The effluents from the first riser 102 and second riser 104, after catalyst disentrainment (refer to FIG. 1), can be fed to a fractionator 208 for separation of any heavy naphtha and heavier oils to yield olefin-rich stream 214. Stream 214 can be pressurized in compressor 216 to a pressure of from about 100 kPa to about 3500 kPa, depending on the separation scheme (an example range is from 100 kPa to 1500 kPa for a depropanizer-first scheme). The pressurized stream 218 can be subjected to treatment as necessary in conditioning unit 220 to remove oxygenates, acid gases and any other impurities from the cracked gas stream to produce a treated gas stream 221, followed by drying in dryer 222. Although the order of fractionation can vary, the dried stream 224 can be fed to depropanizer 226 where the stream can be fractionated into a heavier stream 228 containing C₄ and gasoline components and a lighter stream 230 containing C₃ and lighter components. The heavier stream 228 can be routed to a gasoline splitter 232 where the stream can be separated into a gasoline component stream 234 and a C₄, C₅, and/or C₆ effluent stream 236, which can be recycled to the second riser 104. In one or more embodiments, the heavier stream 228 can be routed to a gasoline splitter 232 where the stream can be separated into a gasoline component stream 234 and a C₄-C₁₀ effluent stream 236, which can be recycled to the second riser 104. Alternately, if there are no C₁₁⁺ components in the heavier stream 228, it can be recycled via line 229 directly to the second riser 104.

The lighter stream 230 from the depropanizer can be compressed in compressor 246 to a pressure of from about 500 kPa to about 1500 kPa to form pressurized stream 248 which can be routed to a cryogenic chill train 250. A light stream 252 can be removed from the chill train as a fuel gas, a product exported from the process, and/or for further processing such as hydrogen recovery or the like. The heavier stream 254 from the chill train can be fed to a series of separators for isolation of olefin streams. The stream 254 can be fed to a demethanizer 256, which produces a light recycle stream 258 and a heavier product stream 260. The light recycle stream 258 can alternatively in whole or in part be a product of the process. The heavier product stream 260 can be routed to a deethanizer 262 where it can be separated into a light component stream 264 including ethylene and a heavier stream 270 including propylene. Stream 264 can be separated into an ethylene product stream 266 and an ethane stream 268 that can be recycled to a steam pyrolysis unit, or stream 264 can be a product of the process. The heavier stream 270 from the deethanizer 262 can be routed to a C₃ splitter 272 where the stream is split into a propylene product stream 274 and propane stream 276 that can be recycled to a steam pyrolysis unit, or the stream can be a product of the process.

The combustion of the coke can be in a common regenerator, such as the regeneration zone 108 depicted in FIG. 1. If coke on the recovered catalyst is insufficient, the regeneration can include combustion of supplemental fuel introduced to the regenerator to maintain a steady state heat balance. Examples of the supplemental fuel include fuel oil (such as kerosene), fuel gas, syngas, or the like.

A coke precursor can be fed to the second riser 104 with the second feed in line 106 at a ratio of from 1 to 40 parts by weight coke precursor to 100 parts by weight fresh light hydrocarbon feed. The coke precursor can include acetylene, alkyl- or allyl-substituted acetylene, (such as methyl acetylene, vinyl acetylene, or the like), a diolefin (such as butadiene), vacuum gas oils, reduced crudes, atmospheric tower bottoms, vacuum tower bottoms, or any mixture thereof. The coke precursor can also include an aromatic hydrocarbon or an aromatic precursor that forms aromatics in the cracking reactor, which can be fed to the second riser 104 with an olefinic feed. In this manner, the feed to the second riser 104 can be paraffinic, and the second riser operating conditions can include a higher temperature, higher catalyst-to-feed ratio, and/or lower hydrocarbon partial pressure relative to the first riser 102. The coke precursor can also include gas oil, which can be fed to the second riser 104 with a paraffinic feed. The second riser 104 operating conditions with the paraffinic hydrocarbon/gas oil coke precursor feed can include a higher temperature, higher catalyst-to-feed ratio, and/or lower hydrocarbon partial pressure relative to the first riser 102. In an embodiment, the process can include preparing the light hydrocarbon feed by partially hydrogenating a diolefin-rich stream to obtain the first light hydrocarbon feed. As an example, the first light hydrocarbon feed in line 105 can include mono-olefins and from 0.05 to 20 or from 1 to 15 wt % diolefins.

In one or more embodiments, coke on the recovered catalyst from the light hydrocarbon feeds can be insufficient by itself to provide for a steady heat-balance. The introduction of the coke precursor can provide additional coke make, so that the combustion of supplemental fuel, otherwise introduced to the regenerator as needed to maintain a steady state heat balance, can be reduced or eliminated. If desired, the introduction of the coke precursor can be controlled at a rate to provide additional coke make to maintain a steady state heat balance without supplemental fuel, or with a given rate of fuel supplementation.

The exothermic methanol-to-olefins reaction in the first riser 102 can generate sufficient heat to maintain a steady state heat balance in the system. In this manner, the introduction of coke precursor to the second riser 104 can be reduced or eliminated.

The dual riser process can condition the gas separated from the first and second effluents 103,109 to remove oxygenates, acid gases, water or a mixture thereof to form a conditioned stream. In one or more embodiments, the first effluent in line 103 and the second effluents in line 109 can be conditioned and recovered together in a common recovery system or in separate recovery systems. The first effluent in line 103 and the second effluents in line 109 can be combined upon exiting the first riser 102 and the second riser 104 and can be treated in the common recovery system. For example, a conditioned stream in line 221 can be separated into at least a tail gas stream, an intermediate stream, and/or a heavy stream. As an example, the tail gas stream can include an ethylene product stream, a propylene product stream, a light stream including ethane, propane, or a mixture thereof As an example, the intermediate stream can include olefins selected from C₄ to C₆ olefins and mixtures thereof. As an example, the heavy stream can include C₆ and higher hydrocarbons. The intermediate stream can be recycled to the second riser 104. The heavy stream can also be recycled to the second riser 104.

A first feed including methanol can be converted to olefins, optionally ethylene and propylene among others, in the first riser. In a methanol-to-olefins process, methanol can be dehydrated on a catalyst to form dimethyl ether (DME). The equilibrium mixture of methanol, DME, and water can then converted to light olefins such as ethylene and propylene. In the process, small amounts of butenes, higher olefins, alkanes and some aromatics can also be produced. For example, first riser reaction temperatures can be from about 200° C. to about 600° C., optionally from about 400° C. to about 550° C.

As used herein, the term “light” in reference to feedstock or hydrocarbons generally refers to hydrocarbons having a carbon number less than 12 and optionally less than 10, and “heavy” refers to hydrocarbons having a carbon number of 12 or more. As used herein, “carbon number” refers to the number of carbon atoms in a specific compound, or in reference to a mixture of hydrocarbons the weight average number of carbon atoms.

As used herein, “naphtha” or “full range naphtha” refers to a hydrocarbon mixture having a 10 volume percent boiling point below 175° C. and a 95 volume percent boiling point below 240° C. as determined by distillation in accordance with the standard method of ASTM-D86; “light naphtha” refers to a naphtha fraction with a boiling range within the range of 0° C. to 166° C.; and “heavy naphtha” refers to a naphtha fraction with a boiling range within the range of 167° C. to 211° C.

As used herein, the term “paraffinic” in reference to a feed or stream refers to a light hydrocarbon mixture including at least 80 wt % paraffins, no more than 10 wt % aromatics.

As used herein, the term “aromatic” in reference to a feed or stream refers to a light hydrocarbon mixture including more than 20 wt % aromatics.

As used herein, the term “olefinic” in reference to a feed or stream refers to a light hydrocarbon mixture including at least 20 wt % olefins.

As used herein, the term “light olefinic naphtha” refers to a naphtha fraction with a boiling range within the range of 0° C. to 166° C. and including at least 20 wt % olefins.

As used herein, the term “heavy olefinic naphtha” refers to a naphtha fraction with a boiling range within the range of 167° C. to 211° C. and including at least 20 wt % olefins.

As used herein, the term “mixed C₄'s,” in reference to a feed or stream, refers to a light hydrocarbon mixture including at least 90 wt % of hydrocarbon compounds having 4 carbon atoms.

As used herein the term “waxy gas oil” refers to a gas oil including at least 40 wt % paraffins and having a fraction of at least 50 percent by weight above 345° C.

As used herein, the term “dual riser” is used to refer to fluidized bed reactors employing two or more risers. While operating complexity and mechanical design considerations can limit the dual riser unit to two risers as a practical matter, a dual riser unit can have three, four or even more risers.

As used herein, reference to a riser temperature refers to the temperature of the effluent exiting at the top of the riser. Because the methanol-to-olefin reactions in the first riser are usually exothermic, the thermal equilibrium of the riser feed (methanol, oxygenates, catalyst) can be lower than the riser exit temperature and the temperature will vary throughout the riser depending on the reactions. Because the cracking reactions in the second riser are usually endothermic, the thermal equilibrium of the riser feed (preheated hydrocarbon, steam and catalyst) can be higher than the riser exit temperature and the temperature can vary throughout the riser depending on the reactions.

As used herein, a “catalyst-to-feed ratio” refers to the weight of catalyst to the weight of feed introduced to the riser. Delta coke and/or coke make refer to the net coke deposited on the catalyst, expressed as a percent by weight of the catalyst. The proportion of steam in a feed refers to the proportion or percentage of steam based on the total weight of hydrocarbon feed to the riser (excluding catalyst).

### EXAMPLES

To provide a better understanding of the foregoing discussion, the following comparative example and prophetic example are provided. All parts, proportions and percentages are by weight unless otherwise indicated.

Comparative Example 1 involved a single riser reactor that used a modified ZSM-5 catalyst to convert methanol to olefins. Reactor yields from the single riser reactor were published in a research paper entitled “High Propylene Selectivity in Methanol to Olefin Reaction over H-ZSM-5 Catalyst Treated with Phosphoric Acid.” See, e.g., Journal of the Japan Petroleum Institute, Vol. 53 (4), pages 232-238, 2010. A set of product selectivities (assumed wt % for purposes of this example and normalized to 100%) at 100% methanol conversion was reported and is shown in Table 1 below. As shown in Table 1, the total ethylene plus propylene concentration was 63.61 wt %.

TABLE 1
(single riser)

| Component | Wt % |
|---|---|
| Methane | 1.89 |
| Ethylene | 8.87 |
| Propane | 0.70 |
| Propylene | 54.74 |
| C4S | 18.65 |
| C5+ | 13.86 |
| Aromatics | 1.30 |
| Total | 100.00 |

Prophetic Example 2 uses a dual riser reactor using a modified ZSM-5 catalyst to convert methanol to olefins. In this simulated example, the same methanol feed that was used in Example 1 is fed to a first riser. Experimental observed yields for C4s and C5+ hydrocarbons using the modified ZSM-5 type catalyst was used as the basis for this prophetic Example 2. See, e.g., Michael J. Tallman & Curtis N. Eng, “Catalytic Routes to Olefins” presented in New Orleans, La. Apr. 7-8, 2008. In this prophetic example, the C4s and C5+ are recycled to the second riser of the reactor. The results are shown in Table 2.

TABLE 2
(dual riser)

| Component | Wt % |
|---|---|
| Methane | 4.48 |
| Ethylene | 15.37 |
| Propane | 2.36 |
| Propylene | 68.27 |
| C4s | Recycled |
| C5+ | Recycled |
| C6+ | 4.65 |
| Aromatics | 4.86 |
| Total | 100.00 |

As shown in Table 2, recycling the C4s and C5+ by-products to the second riser significantly increases the concentration of both ethylene and propylene as compared to the comparative example above that only used a single riser. More particularly, the ethylene concentration increases from 8.87 wt % to 15.37 wt % and the propylene concentration increases from 54.74 wt % to 68.27 wt %. As such, the combined ethylene and propylene in Example 2, as simulated using the dual risers, is 83.64 wt % compared to only 63.61 wt % for Example 1 that used only a single riser.

Embodiments of the present disclosure further relate to any one or more of the following paragraphs:

1. A dual riser fluidized catalytic cracking process, comprising: converting a first feed comprising at least about 50 wt % methanol in a first riser under a first set of process conditions to produce a first effluent enriched in ethylene, propylene, or a mixture thereof, wherein the first effluent comprises at least about 25 wt % dry basis propylene; converting a second feed comprising C₄-C₁₀ light hydrocarbons in a second riser under a second set of process conditions to produce a second effluent enriched in ethylene, propylene, or a mixture thereof; combining the first effluent with the second effluent to produce a mixed effluent; separating the mixed effluent to produce a coked-catalyst and a gaseous product; regenerating the coked-catalyst by combusting the coke in a regenerator to produce a regenerated catalyst; and recycling the regenerated catalyst to the first and second risers.

2. The process of paragraph 1, wherein the first feed further comprises one or more of ethers, oxygenates, or combinations thereof.

3. The process of according to paragraph 1 or 2, wherein the second feed comprises light olefinic naphtha, heavy olefinic naphtha, mixed olefinic C₅s and combinations thereof.

4. The process according to any one of paragraphs 1 to 3, wherein the first and second set of process conditions differ from one another by at least one condition selected from temperature, catalyst-to-feed ratio, hydrocarbon partial pressure, and steam-to-feed ratio.

5. The process according to any one of paragraphs 1 to 4, wherein both of the first and second risers employ one or more catalysts comprising one or more zeolites, and wherein both of the first and second rises employ the same one or more catalysts.

6. The process according to any one of paragraphs 1 to 5, wherein both of the first and second risers employ a zeolite ZSM-5 catalyst.

7. The process according to any one of paragraphs 1 to 6, wherein the second feed further comprises one or more recycle streams recovered from the gaseous product, wherein the one or more recycle streams comprises C₄+ hydrocarbons.

8. The process according to any one of paragraphs 1 to 7, wherein at least about 50 wt % of the first feed is converted to ethylene, propylene, or the mixture thereof in the first riser.

9. The process according to any one of paragraphs 1 to 8, wherein at least about 40 wt % of the second feed is converted to ethylene, propylene, or the mixture thereof in the second riser.

10. The process of claim 1, wherein the first set of process conditions includes a temperature from about 400° C. to about 550° C. and the second set of process conditions includes a temperature from about 590° C. to about 675° C.

11. The process according to any one of paragraphs 1 to 10, wherein regenerating the recovered catalyst further comprises combusting one or more supplemental fuels introduced to the regenerator.

12. The process according to any one of paragraphs 1 to 11, wherein a C4+ product is recovered from the gaseous product and recycled to the second riser.

13. The process according to any one of paragraphs 1 to 12, further comprising: conditioning the gaseous product to remove oxygenates, acid gases, water, or a mixture thereof to form a conditioned stream; separating the conditioned stream into one or more of a tail gas stream, an ethylene product stream, a propylene product stream, a stream comprising ethylene, propylene, or a mixture thereof, an intermediate stream comprising C₄ to C₆ olefins and mixtures thereof, and a heavy stream comprising C₆+ hydrocarbons; and recycling the intermediate stream to the second riser.

14. The process of paragraph 13, further comprising recycling the heavy stream to the second riser.

15. A dual riser fluidized catalytic cracking process, comprising: converting a first feed comprising at least about 60 wt % methanol in a first riser under a first set of process conditions to produce a first effluent enriched in ethylene, propylene, or a mixture thereof, wherein the first effluent comprises at least about 25 wt % dry basis propylene, and wherein at least about 50 wt % of the first feed is converted to ethylene, propylene, or the mixture thereat converting a second feed comprising C₄-C₁₀ light hydrocarbons in a second riser under a second set of process conditions to produce a second effluent enriched in ethylene, propylene, or a mixture thereof, wherein at least about 40 wt % of the second feed is converted to ethylene, propylene, or the mixture thereof; combining the first effluent with the second effluent to produce a mixed effluent; separating the mixed effluent to produce a coked-catalyst and a gaseous product; regenerating the coked-catalyst by combusting the coke in a regenerator to produce a regenerated catalyst; recycling the regenerated catalyst to the first and second risers; separating C4-C10 hydrocarbons from the gaseous product; and recycling the C₄-C₁₀ light hydrocarbons to the second riser.

16. The process of paragraph 15, wherein the first set of process conditions includes a temperature from about 400° C. to about 550° C. and the second set of process conditions includes a temperature from about 590° C. to about 675° C.

17. The process according to paragraphs 15 or 16, wherein both of the first and second risers employ a zeolite ZSM-5 catalyst.

18. The process according to any one of paragraphs 15 to 17, wherein the first feed further comprises one or more of ethers, oxygenates, or any combination thereof and the second feed further comprises light olefinic naphtha, heavy olefinic naphtha, mixed olefinic C₅s, or any combination thereof.

19. A dual riser fluidized catalytic cracking process comprising: converting a first feed comprising at least about 80 wt % methanol in a first riser comprising a zeolite ZSM-5 catalyst under a first set of process conditions to produce a first effluent enriched in ethylene, propylene, or a mixture thereof, wherein the first effluent comprises at least about 25 wt % dry basis propylene, and wherein at least about 50 wt % of the first feed is converted to ethylene, propylene, or the mixture thereof; converting a second feed comprising C₄-C₁₀ light hydrocarbons in a second riser comprising a zeolite ZSM-5 catalyst under a second set of process conditions to produce a second effluent enriched in ethylene, propylene, or the mixture thereof, wherein at least about 40 wt % of the second feed is converted to ethylene, propylene, or the mixture thereof; combining the first effluent with the second effluent to produce a mixed effluent; separating the mixed effluent to produce a coked-catalyst and a gaseous product; regenerating the coked-catalyst by combusting the coke in a regenerator to produce a regenerated zeolite ZSM-5 catalyst; recycling the regenerated zeolite ZSM-5 catalyst to the first and second risers; and fractionating the gaseous product to produce a stream comprising heavy naphtha and a stream comprising olefins.

20. The process of paragraph 19, further comprising: introducing at least a portion of the stream comprising olefins to a depropanizer to produce a stream comprising C4+ hydrocarbons; and recycling the stream comprising C4+ hydrocarbons to the second riser.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

The foregoing description of the invention is illustrative and explanatory of the present invention. Various changes in the materials, apparatus, and process employed will occur to those skilled in the art. It is intended that all such variations within the scope and spirit of the appended claims be embraced thereby.

1-20. (canceled)

21. A dual riser fluidized catalytic cracking process, comprising:
converting a first feed comprising 100 wt % methanol in a first riser under a first set of process conditions to produce a first effluent enriched in ethylene, propylene, or a mixture thereof, wherein the first effluent comprises at least 45 wt % dry basis propylene;
converting a second feed comprising at least 45 wt % C₄-C₁₀ light hydrocarbons in a second riser under a second set of process conditions to produce a second effluent enriched in ethylene, propylene, or a mixture thereof, wherein a weight ratio of the first feed to the second feed is between 1:5 to 5:1;
operating the first riser between 200 degrees C. to 450 degrees C.;
operating the second riser between 400 degrees C. to 900 degrees C.;
heating the second riser using a heat generated by the first riser;
combining the first effluent with the second effluent to produce a mixed effluent;
separating the mixed effluent to produce a coked-catalyst and a gaseous product;
regenerating the coked-catalyst by combusting the coke in a regenerator to produce a regenerated catalyst; and
recycling the regenerated catalyst to the first and second risers.

22. The process of claim 21, further comprising:
conditioning the gaseous product to remove oxygenates, acid gases, water, or a mixture thereof to form a conditioned stream;
separating the conditioned stream into one or more of a tail gas stream, an ethylene product stream, a propylene product stream, a stream comprising ethylene, propylene, or a heavy stream comprising C₄ to C₆ olefins and mixtures thereof, and a heavy stream comprising C₆+hydrocarbons;
recycling the intermediate stream to the second riser; and
recycling the heavy stream to the second riser.

23. The process of claim 21, wherein the second feed comprises light olefinic naphtha, heavy olefinic naphtha, mixed olefinic C₅s and combinations thereof.

24. The process of claim 21, wherein the first and second set of process conditions differ from one another by at least one condition selected from catalyst-to-feed ratio, hydrocarbon partial pressure, and steam-to-feed ratio.

25. The process of claim 21, wherein both of the first and second risers employ one or more catalysts comprising one or more zeolites, and wherein both of the first and second risers employ the same one or more catalysts.

26. The process of claim 21, wherein both of the first and second risers employ a zeolite ZSM-5 catalyst.

27. The process of claim 21, wherein the second feed further comprises one or more recycle streams recovered from the gaseous product, and wherein the one or more recycle streams comprises C4+ hydrocarbons.

28. The process of claim 21, wherein at least 50 wt % of the first feed is converted to ethylene, propylene, or the mixture thereof in the first riser.

29. The process of claim 21, wherein at least 40 wt % of the second feed is converted to ethylene, propylene, or the mixture thereof in the first riser.

30. The process of claim 21, wherein regenerating the recovered catalyst further comprises combusting one or more supplemental fuels introduced to the regenerator.

31. A dual riser fluidized catalytic cracking process, comprising:
converting a first feed comprising 100 wt % methanol in a first riser under a first set of process conditions to produce a first effluent enriched in ethylene, propylene, or a mixture thereof, wherein the first effluent comprises at least 45 wt % dry basis propylene;
converting a second feed comprising at least 45 wt % C₄-C₁₀ light hydrocarbons in a second riser under a second set of process conditions to produce a second effluent enriched in ethylene, propylene, or a mixture thereof, wherein a weight ratio of the first feed to the second feed is between 1:5 to 5:1;
operating the first riser between 200 degrees C. to 450 degrees C.;
operating the second riser between 400 degrees C. to 900 degrees C.;
heating the second riser using a heat generated by the first riser;
combining the first effluent with the second effluent to produce a mixed effluent;
separating the mixed effluent to produce a coked-catalyst and a gaseous product;
regenerating the coked-catalyst by combusting the coke in a regenerator to produce a regenerated catalyst; and
recycling the regenerated catalyst to the first and second risers,
wherein the first riser has a different catalyst-to-feed ratio, partial pressure, residence time, and flow rate than the second riser, and
wherein both of the first and second risers employ a zeolite ZSM-5 catalyst, wherein a catalyst to first feed ratio is between 20:1 to 30:1 and a catalyst to second feed ratio is between 12:1 to 25:1.

32. The process of claim 31, wherein the second feed comprises light olefinic naphtha, heavy olefinic naphtha, mixed olefinic C₅s and combinations thereof.

33. The process of claim 31, further comprising:
conditioning the gaseous product to remove oxygenates, acid gases, water, or a mixture thereof to form a conditioned stream;
separating the conditioned stream into one or more of a tail gas stream, an ethylene product stream, a propylene product stream, a stream comprising ethylene, propylene, or a heavy stream comprising C₄ to C₆ olefins and mixtures thereof, and a heavy stream comprising C₆+ hydrocarbons;
recycling the intermediate stream to the second riser; and
recycling the heavy stream to the second riser.

34. The process of claim 31, wherein the second feed further comprises one or more recycle streams recovered from the gaseous product, and wherein the one or more recycle streams comprises C4+ hydrocarbons.

35. The process of claim 31, wherein at least 50 wt % of the first feed is converted to ethylene, propylene, or the mixture thereof in the first riser and wherein at least 40 wt % of the second feed is converted to ethylene, propylene, or the mixture thereof in the first riser.
